(19) United States
(12) Patent Application Publication
(10) Pub. No.: US 2026/0272366 A1
(43) Pub. Date: Sep. 17, 2026

(54) NON-INVASIVE NEURAL INTERFACE AND METHOD OF MAKING THE SAME

(71) Applicant: Board of Trustees of Michigan State University, East Lansing, MI (US)

(72) Inventors: Vittorio MOTTINI, East Lansing, MI (US); Jinxing LI, Okemos, MI (US)

(73) Assignee: Board of Trustees of Michigan State University, East Lansing, MI (US)

(21) Appl. No.: 19/168,651

(22) PCT Filed: Mar. 29, 2024
(86) PCT No.: PCT/US2024/022163

§ 371 Date: Sep. 24, 2025

Related U.S. Application Data
(60) Provisional application No. 63/457,879, filed on Apr. 7, 2023.

(51) Int. Cl. A61B 5/389; A61B 5/268; A61B 5/296

(52) U.S. Cl. CPC A61B 5/389; A61B 5/268; A61B 5/296; A61B 2562/125; A61B 2562/164

(57) ABSTRACT

A surface EMG sensor and method of making the same includes a substrate and an electrode array disposed on the substrate. The electrode array has a conductive polymer, an ionic liquid (IL), a water-based polyurethane matrix (WPU) forming interconnects and sensing pad. An encapsulation layer is disposed on the interconnects of the electrode array. The electrode array is formed of PEDOT:PSS.

### CROSS-REFERENCE TO RELATED APPLICATIONS

This application is a national phase entry of PCT international patent application serial no. PCT/US2024/022163, filed on Mar. 29, 2024, which claims the benefit of U.S. Provisional Application No. 63/457,879, filed on Apr. 7, 2023. The entire disclosures of the above applications are incorporated herein by reference.

### FIELD

The present disclosure relates to surface electromyography and, more specifically, to a non-invasive neural interface and method of making the same for performing surface electromyography.

### BACKGROUND

This section provides background information related to the present disclosure which is not necessarily prior art.

Electromyography (EMG) is a technique used to measure the electrical activity of muscles, typically through microneedle electrodes implanted in the muscle through the skin. It has been widely employed in biomedical research and clinical settings, as it allows for assessing muscle function and can provide valuable insights into the health and functioning of the nervous system. EMG signals are produced by the electrical activity of muscles and are caused by the firing of muscle fibers in response to neural signals. By measuring the EMG signals, it is possible to identify the specific muscles being activated and the intensity of the muscle activity. This information can be used to evaluate muscle function, diagnose neuromuscular disorders and monitor the effectiveness of rehabilitation techniques.

However, current EMG and surface EMG biosensors have several limitations that have hindered their effectiveness and widespread use. One major drawback is that they are rigid, which can be uncomfortable and inconvenient for users, especially when worn for extended periods. The lack of flexibility and stretchability of the sensors makes it difficult to obtain reliable, accurate, and comparable measurements on individuals with different skin topographies or conditions, such as wrinkles or hair. In addition, EMG biosensors routinely used in clinical settings for neuromuscular connection monitoring are invasive, requiring the insertion of electrodes directly into the muscle tissue which can be painful and cause discomfort or irritation to the skin. Invasive EMG biosensors are also unsuitable for certain populations, such as children or elderly individuals, who may have more sensitive or fragile skin. Surface EMG electrodes allow overcoming the invasiveness of conventional EMG electrodes. However, these are rigid and can discomfort the subjects being tested.

Moreover, the imperfect interface between electrodes and skin adds noise and artifacts to the recording, making these devices sub-optimal. Another critical aspect of surface EMG sensors is their impedance, which refers to the sensor's resistance to the flow of electrical current. A high impedance can introduce noise and interference into the measured signal, leading to inaccurate and unreliable data. A low impedance is essential for accurately detecting and amplifying the small electromyographic signals muscles produce. Conventional EMG technologies have often struggled to achieve a low enough impedance, especially when the sensors are worn over large body areas. This limitation has further restricted the range of applications and made it difficult to obtain high-quality data, especially in situations where the signal-to-noise ratio is low.

Another limitation of current EMG biosensors is their lack of ability to monitor large areas of the body. Many EMG biosensors are designed to measure the activity of a single muscle or muscle group, which limits their ability to provide a comprehensive assessment of muscle function. This can be particularly problematic for studying movement or activities that involve multiple muscles working together, as it is difficult to assess the contribution of each muscle accurately. In order to monitor the activity of multiple muscles simultaneously, multiple EMG biosensors are often required, which can be inconvenient and cumbersome for the user.

To improve the effectiveness of wearable technologies, it is essential to create flexible, stretchable electronics that can conform to the skin features without discrimination. In recent years, researchers have developed flexible surface electromyography (sEMG) sensors using polymeric materials to overcome the limitations of rigid electronics. However, these wearable devices have difficulty conforming to the microscopic curvature of the skin, resulting in microscale air pockets that hinder the formation of ideal electrical and physical contacts.

### SUMMARY

This section provides a general summary of the disclosure and is not a comprehensive disclosure of its full scope or all of its features.

A new large-area, high-density surface EMG (HD-sEMG) electrode array using intrinsically stretchable materials is provided. This device, made with conductive composites described below allows for better conformality, ease of use, comfort, and the ability to record signals from various skin morphologies. Each solution has somewhat varying properties and may be used in different conditions. The present HD-sEMG electrode array outperformed traditional rigid electronics in electrode impedance, signal-to-noise ratio, and EMG signal amplitude for all skin morphologies tested. In addition to providing sEMG measurements, the present disclosure also enables the generation of sEMG maps from large body areas and the control of robotic prosthetics and 3D arm models in virtual reality environments.

In one aspect of the disclosure, A surface EMG sensor and method of making the same includes a substrate and an electrode array disposed on the substrate. The electrode array has a conductive polymer, an ionic liquid (IL), a water-based polyurethane matrix (WPU) forming interconnects and sensing pad. An encapsulation layer is disposed on the interconnects of the electrode array. The electrode array is formed of PEDOT:PSS.

In another aspect of the disclosure, a method of forming an electromyography (EMG) sensor includes forming an electrode solution, drying the electrode solution to form a dried electrode solution, forming an electrode array, transferring the electrode array to a substrate, plasma etching the electrode array and encapsulating the electrode array with an encapsulation layer so sensing pads are exposed at the electrode array to form an encapsulated EMG array to form an encapsulated EMG array.

In another aspect of the disclosure, a method of forming an electrode includes depositing a mixture of poly(3,4-ethylenedioxythiophene):polystyrene sulfonate (PEDOT:PSS), an ionic liquid (IL), a water-based polyurethane matrix (WPU) and glycerol to form an electrode layer on a first substrate layer that forms interconnects and sensing pads, encapsulating the interconnects of the electrode layer with an encapsulation layer without encapsulating the sensing pads with the encapsulation layer to form exposed sensing pads and depositing PEDOT:PSS glycerol on the exposed sensing pads.

One advantage of the electrodes developed in this manner is that the ability to interface with multiple electro-biopotential applications including but not limited to EKG, ECG, EEG and EMG. Another advantage is that the electrodes can stay on the skin surface for several days rather than minutes as in other known devices.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A is a view sensor having an electrode array.

FIG. 1B is a close up view of a bent finger having the electrodes formed thereon.

FIG. 1C is a close up view of a straight finger having the electrodes formed thereon.

FIG. 2A is a plot of tensile strength for different formulations of electrodes.

FIG. 2B. is an enlarged view of stretched electrodes having cracks therein.

FIG. 2C is a plot of resistance versus strain for a plurality of electrode formulations.

FIG. 2D is a plot of impedance versus type of electrodes at different frequencies.

FIG. 3 is a flowchart of a method of forming electrodes.

Corresponding reference numerals indicate corresponding parts throughout the several views of the drawings.

### DETAILED DESCRIPTION

Example embodiments will now be described more fully with reference to the accompanying drawings.

In the following description, different solutions are mentioned. Some of the solutions have compositions that may vary depending on the conditions in which they are to be used.

Solution W refers to an electrode solution having a mixture with conductive polymer such as poly(3,4-ethylenedioxythiophene):polystyrene sulfonate (PEDOT:PSS), ionic liquid (IL), and a water-based polyurethane matrix (WPU).

Solution WG is the electrode Solution W with an amount of glycerol added thereto.

Solution WGP is a coating used on the electrodes. The coating is a layer placed on top of the sensing pads formed of Solution W or Solution WG and is composed of PEDOT:PSS and glycerol.

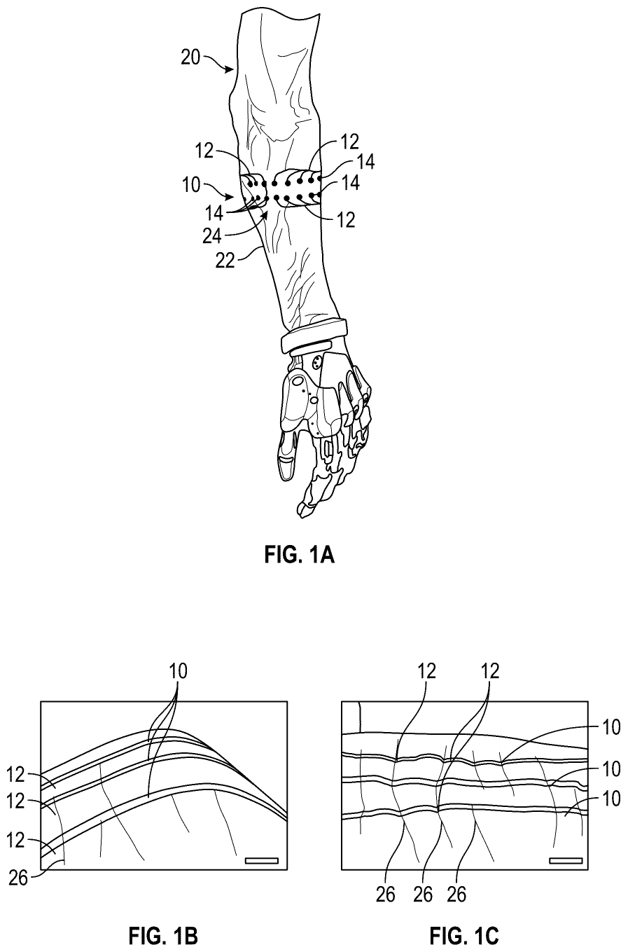

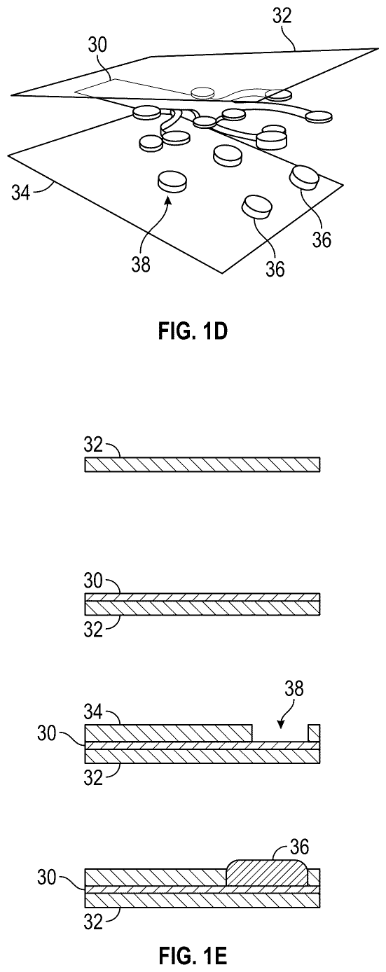

Referring now to FIG. 1A, an electrode array 10 has interconnects 12 and sensing pads 14. The electrode array 10 is disposed on a skin of a body part 20 such as an arm 22. However, other body parts may be used as described below. To address the deficiencies of the prior art set forth above, the electrode array 10 forms stretchable electronics for use as a surface electromyography (sEMG) sensor 24. The electrode array 10 allows the devices to conform seamlessly to the skin features and provide more accurate monitoring of biosignals. The electrode array 10 provides a flexible, stretchable, and low-impedance array using intrinsically stretchable materials and conductors, which can seamlessly conform to the underlying skin features and provide large-area and high-density surface EMG monitoring and mapping. The electrode array 10 can map the muscle motion pattern of body parts 20 such as the arm and conform to complex skin morphologies such as hair and wrinkles.

Materials for the sEMG sensor 24 and the electrode array 10 include a first mixture of conductive polymer such as poly(3,4-ethylenedioxythiophene):poly(styrene sulfonate) (PEDOT:PSS), an ionic liquid (IL), and a water-based polyurethane matrix (WPU), with biocompatible glycerol introduced to modulate the mechanical properties while maintaining high conductivity of the conductive polymer. Additionally, the composite mixture is solution processable that allows the electrode array 10 to be patterned in various shapes to adapt to the body part under investigation.

Referring now also to FIG. 1B and FIG. 1C, the electrode array 10 in this example is a micro-scale resolution laser-patterned array conforming to micro features of the skin of the body part 20 such as a bent finger 26, which is illustrated in a bent position in FIG. 1B and in FIG. 1C in an extended position. The intrinsic stretchability and low thickness allow the electrode array 10 to conform seamlessly to the underlying skin features, such as the wrinkles 26 in FIG. 1C.

Referring now to FIGS. 1D and 1E, the electrode array 10 adapts to various skin morphologies, enabling real-time monitoring of sEMG signals and remote control of prosthetics. The SEMG sensor 24 is an epidermal biopotential sensor that comprises a multilayered assembly of intrinsically stretchable materials. A stretchable conductive polymer composite electrode layer 30, exhibiting low impedance, is encapsulated between a first layer substrate layer 32 and a second encapsulation layer 34 to form an encapsulated EMG array or sensor. The layers 32, 34 are formed of a thin, stretchable polyurethane membrane coated with an acrylic adhesive layer called “artificial skin” (AS). The electrode layer 30 forms the interconnects 12 and one layer of the pads 14. The substrate layer 32 is the layer on which the electrode layer 30 is formed. The encapsulation layer 34 is formed on the interconnects 12 without encapsulating the sensing pads 14 to form exposed sensing pads as indicated by the openings 38. In FIG. 1D, the opening 38 is formed at the edge of the layer 24 directly adjacent to the coating 36. The exposed sensing pads 14 are coated with the coating 36 of stretchable composite to enhance their electrical performance. Electrical connections between the electrode array 10 and the recording electronics are established through flat-flexible cables (FFCs) not illustrated. Further details of the formation of the electrode array 10 and the sensor 24 are set forth below.

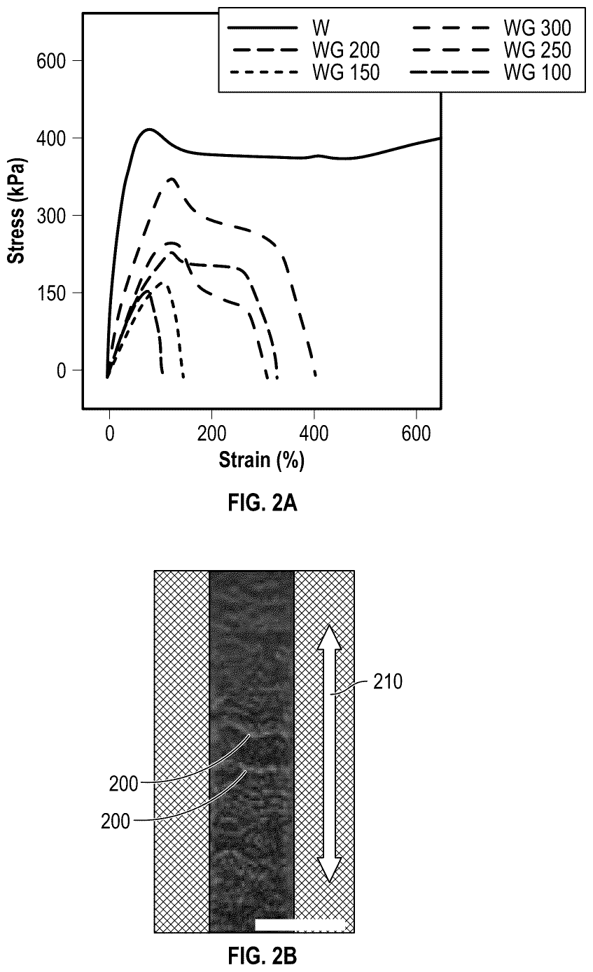

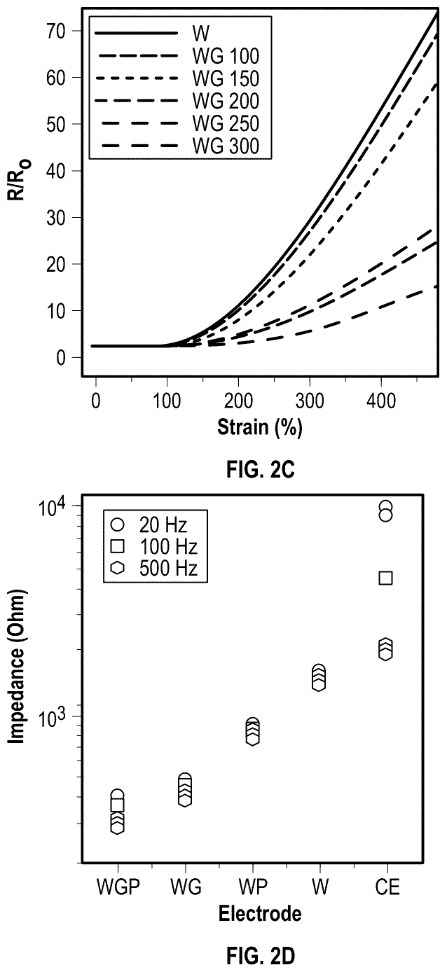

Referring now to FIG. 2A, a stress versus strain (or tensile strength plot for determining the electromechanical properties of the electrically active stretchable composite to evaluate device performance is set forth. Tensile stress measurements reveal that the PEDOT:PSS/IL/WPU composite, referred to as Solution W, demonstrates a significantly higher strain to failure (~1500%) compared to previously reported PEDOT:PSS-based composites. Adding glycerol in various proportions such as WG100, WG150, WG 200, WG250 and WG300 are set forth and indicate the ratio of Solution W to glycerol, i.e., WG100 has a ratio of solution W to glycerol of 100:1. Glycerol is a disorder-inducing agent, resulting in a more uniform and isotropic thin film as observed by an electron microscope. The impact of glycerol addition on the composite was studied by synthesizing and characterizing various formulations with different glycerol-to-Solution W ratios, ranging from 1:1 to 1:300. Composite formulations containing Solution W and Glycerol have been named Solution WG.

Composites with a glycerol-to-Solution W ratio higher than 1:50 could not maintain their shape, while those with ratios between 1:50 and 1:100 exhibited poor adhesion between the thin films and AS. However, composites with ratios lower than 1:100 maintained a freestanding form and were easily transferred to the AS substrate.

The mechanical properties of the different Solution WG formulations were also explored. As expected, increasing the glycerol content in the composite compromised its mechanical properties. Nevertheless, formulations with ratios lower than 1:200 exhibited no fracturing up to 250% elongation. Glycerol, functioning as a lubricant, enabled the composite to accommodate elongation and prevent the formation of bands 200 transverse to a stretching direction 210, as depicted in FIG. 2B.

Referring now to FIG. 2C, the electrical performance under mechanical deformation by analyzing resistance changes upon stretching. All tested composites exhibited a less than 50-fold resistance increase when stretched up to 400%. Solution W mixed with glycerol at a 250:1 ratio, referred to as Solution WG250, displayed optimal performance, with a less than 25-fold resistance increase when stretched up to 600% and only a 2-fold resistance increase when stretched up to 300%.

Moreover, pristine PEDOT:PSS shows a 30-fold increase in resistance under 8% uniaxial strain, while adding glycerol reduces the increase to a 3.9-fold increase. Solution W exhibited less than a 5-fold increase in resistance under 100% uniaxial strain. In comparison, adding glycerol resulted in Solution WG exhibiting only a 27% increase in resistance when stretched up to 100%.

The electromechanical fatigue behavior of the composites was also observed. Solution W and Solution WG250 samples, both freestanding and substrate-supported, underwent cyclic loading while resistance measurements were taken. It was demonstrated that both freestanding and substrate-supported composites exhibit a decrease in resistance upon cycling. Solution WG250 recovers its initial resistance after about 50 cycles, displaying strain-insensitive resistance. The cycling characteristics also revealed that the addition of glycerol makes the composite softer; however, when supported by a substrate, the mechanical properties of the composite have minimal impact on the final device assembled with the substrate-supported material.

Electrode impedance is another factor when measuring surface electromyography (sEMG) signals. Surface EMG detection in space and time. Electrode impedance influences the quality and accuracy of sEMG signals, with high impedance causing interference and leading to inaccurate or unreliable results. In contrast, low-impedance electrodes can provide a more stable and accurate sEMG signal.

Various factors can affect electrode impedance, such as electrode material, the distance between the electrodes and the skin, and skin condition. To ensure accurate sEMG measurements, using low-impedance electrodes improves accuracy.

The surface area enhancement and morphology of PEDOT:PSS allow for increased charge injection per unit area at the electrode-electrolyte interface, resulting in significantly lower electrochemical impedance. Commercially available dry electrodes (CE) for surface EMG recording are typically made of rigid Ag/AgCl disks. When tested in a PBS solution, these electrodes exhibited an average impedance at 100 Hz of approximately 4.4 kΩ. Solution W electrodes demonstrated an impedance of 1.5 kΩ at 100Hz, a nearly 3-fold reduction compared to Ag/AgCl electrodes. Meanwhile, Solution WG exhibited an even more substantial reduction in impedance at 100 Hz, down to 430 Ω, more than a 10- fold reduction compared to commercial electrodes.

The conductive sensing pads 14 were treated by drop-casting a 1:1 PEDOT:PSS/glycerol solution to further enhance the electrode-skin interface. After solvent evaporation, the solution formed a freestanding gel conductor that further reduced electrode impedance by creating a softer interface between the skin and the electrode. The treated materials are referred to as Solution WP and Solution WGP for the treated electrodes made with Solution W and Solution WG, respectively. At 100 Hz, Solution WG exhibited an impedance of 800 Q and Solution WGP of 215 Q, each corresponding to an almost 50% reduction in impedance compared to the uncoated composites.

Comparing the average absolute values of the impedance within the frequency interval of interest for sEMG signals (20-500 Hz), it was found that when compared to the impedance value of commercially-available electrodes (CEs), Solution W and WG electrodes showed a 70% and 87% lower impedance, while, when the electrodes were coated with PEDOT:PSS/Glycerol to give Solution WP and WGP, their impedance further decreased by 96% and 98%, respectively.

Considering the significantly lower impedance, improved behavior upon elongation, and the fact that human skin typically cannot stretch more than 25.45±5.07% under normal conditions, Solution WG and Solution WGP were employed to record sEMG signals.

As illustrated best in FIG. 1D, large area, high-density sEMG arrays were made by vertical integration of four functional layers including a bottom substrate 32 of AS, the electrode layer 10 of Solution WG, an encapsulation layer 32 also of AS, and a final functionalization coating layer 36 deposited on the exposed sensing pads. The composition of the coating layer is PEDOT:PSS and glycerol. The developed fabrication method (with a minimal feature size of 50 μm in the x-y plane by laser engraving) enabled the combined function of high-density and large area fabrication. Moreover, the solution-processable conductive composite and the fabrication based on laser patterning allow customized large-area sEMG arrays. The as-fabricated, low-impedance, extremely thin (less than 70 μm), biomechanically compatible, stretchable, and conformal 32-channel array has a narrow impedance amplitude distribution.

Using the vertical assembly, an extremely thin, biomechanically compatible, stretchable, and conformal sEMG sensor 24 array allows for a stable and intimate interface between the skin-like biosensor and the underlying tissue.

Next, the mechanical, electrical, and electromechanical properties of the composites were studied. FIG. 2A shows stress-strain curves of the free-standing composite films made in Solution W and Solution WG.

To evaluate and compare the electrical properties of Solution W, WP, WG, and WGP, their electrochemical impedance and cyclic voltammetry were measured.

From the comparison of the average values within the frequency interval of interest for sEMG signals (20-500 Hz) shown in FIG. 2D, when compared to the impedance value of CEs, Solution W and WG electrodes showed a 70% and 87% lower impedance.

Moreover, when the electrodes were coated with PEDOT:PSS/Glycerol to give Solution WP and WGP, their impedance further decreased by 96% and 98%, respectively.

The addition of glycerol in the composite formulation, under material stretching to 200%, limits the formation of aligned bands orthogonal to the stretching direction. The effect is a conductor more robust to stretching, also confirmed by the resistance change upon elongation.

Solution W electrodes showed an increased slope in the plot of normalized resistance as a function of the elongation percentage when compared to Solution WG.

The electromechanical properties of Solution W and Solution WG were compared by evaluating the relative change in resistance (R/RO) under tensile ϵ (FIG. 2C). The increase in resistance caused by uniaxial strain was plotted to show two regimes for both composites: up to 100% elongation, both materials showed a slower resistance increase, with Solution W showing a 65% increase and Solution WG a 27% increase.

The second regimen, from 100% elongation to material failure, presents a steeper slope. Solution W showed a 160-fold resistance increase upon stretching up to 900% while solution WG showed a 120-fold increase upon stretching up to 900% and an almost 200-fold increase when stretched up to 1500%. There seems to be a correlation between good electromechanical performance and low E and high ϵf, as the relative resistance of the softer composite decreases less with ϵ. This can be understood by considering the addition of disorder introduced by glycerol, allowing for a softer and more homogeneous material. Glycerol, acting as a lubricant between the PEDOT:PSS chains, preventing their aggregation and consequent composite embrittlement.

Next, the effect of stretching on the electrode impedance was also investigated. All the four different electrode types showed a less than 2-fold increase in impedance under 100% elongation. Specifically, Solution W, the one having the higher impedance among the stretchable electrodes, showed an 80% increase in the impedance absolute value. Solution WP, instead, showed a 67% increase. Solution WG, however, showed only a 60% increase in impedance, again confirming that the addition of glycerol in solution makes the electrodes more resistant to elongation. Lastly, Solution WGP, the formulation already showing the lowest impedance among the ones synthesized in this study, only showed a 38% increase in impedance under 100% uniaxial strain.

From the results obtained during mechanical and electrical characterization, Solution WG and WGP were chosen as the electrodes materials to use during EMG measurements and to then compare to commercially available electrodes.

The electrodes were used to monitor sEMG signals of the biceps brachii of individuals with different skin characteristics. The biceps brachii was chosen as the muscle to be tested due to its relatively large area and for the simplicity of its flexion. The three different electrode types (CE, Solution WG, and Solution WGP) have been tested by recording a single channel sEMG test. The electrodes of 1 cm in diameter were placed 1 cm apart, symmetrically with respect to the peak of the contracted muscle to allow for a fair comparison with CE. Solution WG and WGP electrodes were applied to the skin as fabricated, being the AS adhesive. In contrast, CE electrodes are not adhesive, and they have been secured in place by a layer of AS after placement on the skin and prior to testing.

For all three electrode configurations, a commercially available Ag/AgCl reference electrode was used to allow for the comparison of the signals. The reference electrode for bipolar EMG recording should be placed on the skin above a muscle not involved during the contraction to be measured. For this reason, the reference electrode was placed on the top of the hand for all participants.

To study the electrode behavior to different skin morphologies and to investigate the devices' fairness, different conditions were tested and compared. The electrodes performances in sEMG recording were tested by comparing the signal detected coming from people having wrinkles on their skin or from people with smooth skin. The objective was to evaluate the electrodes performance between individuals with skin having morphological characteristics of old and young age. Next, hairy skins and shaved skins were compared to investigate the ability of the electrodes to conform to rough underlying skins to model different populations. Lastly, the signal recorded during static biceps contraction and one recorded while moving were compared to quantify motion artifacts and evaluate the devices' performances in their intended use.

After electrodes placement, they were connected to the recording system (Intan Technologies™) and the sEMG signal was recorded. The recording instrument also has impedance measurement capabilities, and these were used to compare the electrode-skin impedance for the different electrodes' configurations and different skin characteristics.

After data collection, processing and analysis, from these experiments the electrodes performances between individuals representative of young age versus old age, commercial dry EMG electrodes showed a 77.2% reduction in signal amplitude (p-value=0.05), a 13.85% increase in skin-electrode impedance (n.s) and a 38.3% reduction in SNR (p-value=0.005). The results confirmed that commercial electrodes cannot guarantee the same accuracy and performances for people belonging to different age groups.

Electrodes realized in Solution WG, displayed a 29.5% decrease in signal amplitude when comparing recordings between young and old skin characteristics (p-value=0.05). The electrode-skin impedance value increased by 16% (n.s.), while the SNR decreased from 16 to 4, corresponding to a 75% reduction (p-value=0.005). These results confirm that flexible electrodes do offer an improvement on rigid electronics but still do not allow for proper signal reliability.

Lastly, soft electrodes fabricated with Solution WGP, recorded changes of signal amplitude, electrode-skin impedance, and SNR from young to old skin morphologies of only 23% (p-value=0.005), 26% (n.s), and 53% (p-value=0.005), respectively. These results confirm that soft and stretchable electronics allow for better signal reliability and fairness when interfacing skins of people from different age groups. A possible explanation for the still present change in signal features between smooth skins and wrinkled ones can be attributed to the muscle state. In general, younger individuals have healthier and stronger muscles that weaken with the progression of age. This could explain the difference in signal amplitude and signal-to-noise ratio, moreover, this hypothesis is strengthened by the fact that the same effect is not seen when comparing the signals collected from smooth skins and hairy ones.

These results confirm the hypothesis that stretchable bioelectronics, thanks to its ability to seamlessly conform to the underlying features of the skin, allow for improved, more consistent, and accurate measurements, allowing for more fair electronics for populations of different ages.

Electrodes performances were studied by comparing the features extracted from the sEMG recordings, between hairy skins and shaved skins.

The performance of dry electrodes changed dramatically. Comparing hairy skins with shaved ones, they showed a 6% increase in average electrode-skin impedance (n.s), a 60.5% decrease in signal amplitude (p-value=0.005) and a 77.4% decrease in SNR (p-value=0.005). This evidence further validates the hypothesis that dry electrodes are not suitable to be employed in the larger population. Solution WG electrodes, instead, showed a 184.3% increase in electrode-skin impedance (p-value=0.5), a 30% decrease in signal amplitude (p-value=0.05), and a 47% in SNR (p-value=0.005). These values further confirm that flexible electronics offer significant improvements when compared to rigid ones. However, when employed on skins having rougher morphologies (such as hairs or wrinkles), they do not yet offer a drastic advantage. Lastly, electrodes made with Solution WGP, when comparing sEMG signals from smooth skins and hairy skins, showed a 15.5% (n.s.), 6.1% (n.s.), and 0.5% (n.s.) decrease in electrode-skin impedance, signal amplitude, and SNR, respectively.

In summary, these results show that commercially available dry electrodes offer somewhat consistent results when employed on smooth skins but when used to detect sEMG signals from skins with pronounced morphological features, do not show consistent or reliable performances. Electrodes realized in Solution WG, instead, have shown good signal amplitudes and lowered impedance. However, their signal-to-noise ratio remained low for wrinkled or hairy skins. Lastly, soft electrodes realized in Solution WGP allow to obtain reliable and consistent results from multiple skin conditions, making this a suitable material for fairer and more inclusive biomedical applications.

Next, the effect of movement on signal quality was investigated. The study participants were asked to perform arm contractions while sitting or while freely moving to quantify motion artifacts.

Motion artifacts based on the change of signal-to-noise ratio (SNR), on the assumption that motion artifacts would present as stronger noise in the EMG signal background. Dry electrodes showed an SNR variation between static condition and motion of- 44.37% (p-value=0.005), Solution WG of −21.79% (p-value=0.05) and Solution WGP of only-7.64% (n.s).

The electrode array 10 has then been employed for sEMG mapping. The 2D array composed of 32 monopolar sEMG sensors allows simultaneous recording of the electromyographic signal of multiple muscles. Bipolar surface electromyography (sEMG) signals, which reflect the activity of multiple motor units (MU) within a specific muscle area have limitations in terms of spatial resolution. The amplitude of these signals can vary in parallel and perpendicular directions due to factors such as proximity to innervation zones and tendons and the propagation properties of the conductor volume. Single-channel approaches, which do not consider the physical propagation of the potential, may not accurately reflect important spatial aspects of MU activity and may lead to incorrect conclusions. In contrast, electrode arrays allow for the study of sEMG in both the temporal and spatial domains, providing new insights into the neuromuscular system and myoelectric control.

Recent research has also shown that muscles do not activate uniformly, with different regions activated differently depending on joint position, and strength. This non-uniform activation may be related to different fiber-type bundles within the muscle, each following different recruitment strategies (As a result, the amplitude information provided by a single bipolar channel is highly dependent on electrode location and does not allow for tracking of inhomogeneities in muscle activation.

High-density sEMG (HD-EMG), recorded using 2D arrays of closely spaced electrodes can provide a more significant amount of information related to the tracking of skin surface areas with maximal EMG amplitude and a better estimation of muscle activity.

The stretchable bioelectronics platform's flexibility and functionality were demonstrated by connecting the HD-sEMG electrode array in Solution WGP to a wireless amplifier produced by Mentalab LLC. The Mentalab Explore records and sends the sEMG signal in real-time to a computer. The recorded sEMG signals were processed and features were extracted using a custom algorithm. The classified motion was then sent to an Arduino-controlled robotic arm. The stretchable electrode array, with low unit-area impedance, was able to resolve sEMG propagation dynamics with high spatial resolution. In addition, the integrated EMG signals over time were distinct enough to differentiate various hand gestures in a highly reproducible manner, achieving an average accuracy of 97.7% in gesture differentiation.

This preliminary proof of concept highlights the platform's broad applicability in fields requiring body-machine interfaces such as remote interventions, surgery, prosthetic control, and VR gaming and control. The ability to wirelessly control a prosthetic arm using the recorded sEMG signals demonstrates the technology's potential to improve the quality of life for individuals with limb loss or other mobility impairments significantly. The low-latency and reliable transmission of sEMG signals ensure precise control of the prosthetic arm, further enhancing its practical utility. Notably, the EMG signal was collected using the wireless amplifier produced by Mentalab, ensuring reliable transmission with low latency for precise control of the prosthetic arm.

Wireless prosthetic control through the stretchable bioelectronic platform is a significant achievement with promising implications. The demonstration highlights the versatility of the platform and brings hope for individuals with mobility impairments. The potential of this technology extends to various fields, including remote robotic control for rescue teams, surgery, and VR gaming and control. The stretchable bioelectronics platform's ability to wirelessly control a prosthetic arm has the potential to revolutionize the field of prosthetic control and open up new possibilities for individuals with mobility impairments. Finally, this technology will allow for fairer and more inclusive biomedical technologies.

The performance of various electrode types for surface electromyography (sEMG) recordings on human muscles with diverse skin morphologies and motion artifacts was investigated. Commercially available dry electrodes and flexible electrodes made with Solution WG face considerable limitations in reliably detecting sEMG signals on skin with pronounced morphological features, such as wrinkles and hairs. In contrast, stretchable soft electrodes made with Solution WGP exhibit significantly improved signal reliability and fairness, positioning them as a more suitable candidate for inclusive biomedical applications. The low electrode impedance achieved by the Solution WGP electrodes plays a crucial role in enhancing the sEMG signal quality, as it minimizes noise and interference, enabling the accurate detection and amplification of the weak electromyographic signals produced by muscles.

Furthermore, a high-density and large-area stretchable electrode array using Solution WGP was constructed. This innovative electrode array permits high-density sEMG recordings even on moving subjects, offering valuable insights into the neuromuscular system and myoelectric control. The flexibility provided by the fabrication method allows for the creation of custom and large-area sEMG arrays, making it adaptable to various applications and individual needs. As a result, this stretchable electrode array has the potential to revolutionize the field of sEMG recording and lead to advances in several areas, such as remote interventions, surgery, prosthetic control, and virtual reality (VR) gaming and control. The successful integration of this electrode array with a prosthetic arm serves as a promising proof of concept for the broad applicability of this technology in fields requiring body-machine interfaces. Ultimately, this invention is poised to revolutionize the field of sEMG recording and contribute to the development of more inclusive biomedical technologies.

To demonstrate the practical applications of the stretchable bioelectronic platform of the present disclosure, the high-density sEMG electrode array in Solution WGP was connected to a wireless amplifier, which was then employed to control a prosthetic arm. Using machine learning algorithms, an impressive 97.7% accuracy in gesture recognition was achieved, surpassing the performance of known commercial electrodes. This proof of concept highlights the platform's versatility and applicability in fields requiring body-machine interfaces. The ability to wirelessly control a prosthetic arm using the recorded sEMG signals showcases the technology's potential to significantly improve the quality of life for individuals with limb loss or other mobility impairments. Moreover, the low-latency and reliable transmission of sEMG signals ensure precise control of the prosthetic arm, further enhancing its practical utility.

In addition to prosthetic control, the stretchable bioelectronics platform can be applied to various other fields, including remote robotic control for rescue teams, surgery, and VR gaming and control. The platform's ability to wirelessly control a prosthetic arm opens up new possibilities for individuals with mobility impairments, promoting fairer and more inclusive biomedical technologies. The high-density sEMG (HD-EMG) recorded using 2D arrays of closely spaced electrodes can provide crucial information related to the tracking of skin surface areas with maximal EMG amplitude and a more accurate estimation of muscle activity, leading to improved diagnostics and treatment planning.

Stretchable bioelectronics made with Solution WGP offer a reliable and inclusive platform for sEMG recordings, with significant potential in the development of body-machine interfaces for various applications. The stretchable soft electrodes and the high-density, large-area electrode array enable high-density sEMG recording, even on moving subjects, allowing for more accurate and detailed analyses of muscle activity. The successful integration of this electrode array with a prosthetic arm serves as a promising proof of concept for the broad applicability of this technology in fields requiring body-machine interfaces. The superior performance of the Solution WGP electrodes in terms of signal quality, adaptability to diverse skin conditions, and resilience to motion artifacts, combined with the high-density sEMG mapping capabilities and wireless prosthetic control, position this technology as a groundbreaking advancement in the field of electromyography and human-machine interfaces. Ultimately, the electronic array 10 of the present disclosure is poised to revolutionize the field of sEMG recording and contribute to the development of more inclusive biomedical technologies.

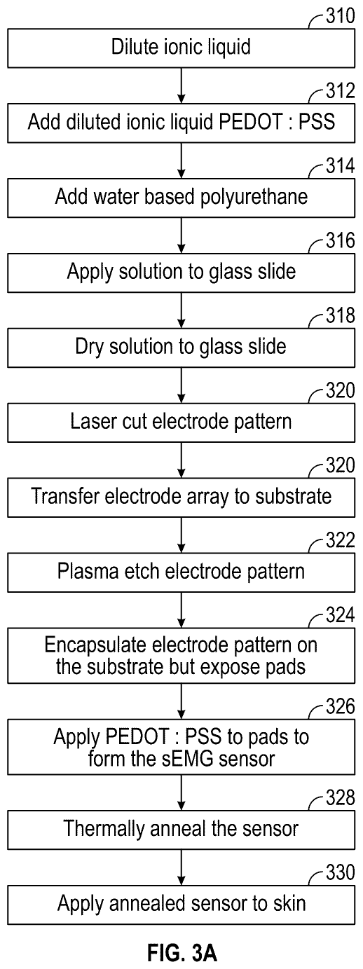

Referring now to FIG. 3, the Solution W was prepared as set forth below.

An aqueous dispersion of PEDOT:PSS (Clevios™ PH1000; solid content=1.1-1.3 wt %; weight ratio of PEDOT to PSS≈1:2.5; pH~2) from Heraeus was used. A waterborne polyurethane (WPU) dispersion (ALBERDINGK® U4101; solid content≈39-41 wt %; elongation at break~1400%; pH=7.0-8.5) from Alberdingk Boley was used. ALBERDINGK® U4101 is an aqueous, anionic dispersion of an aliphatic polyether-polyurethane without free isocyanate groups. 1-Ethyl-3-methylimidazolium coordinated with ethyl sulfate (EMIM:ESO₄) from Sigma-Aldrich was used. Glycerol and an ammonia hydroxide solution 25% from Sigma-Aldrich was used. Tegaderm™ was purchased from 3M™ as the AS or substrate and encapsulation layer. All chemicals were used as received without further purification.

To prepare the composite material for the sEMG electrode array 10, the ionic liquid (ethyl-3-methylimidazolium:ethylene sulfate (EMIM:ES) was diluted to 1.48 wt % with deionized water in step 310 before being added to PEDOT:PSS dispersions in step 312. The ionic liquid solution was added dropwise to the PEDOT:PSS dispersion in a ratio of 15:25, and the mixture was stirred at 400 rpm for more than 12 hours. Before adding a waterborne polyurethane (WPU) dispersion, 0.15 wt % of an ammonia solution was added with respect to the PEDOT:PSS dispersion. In step 314 the WPU was added to the solution to obtain a final ratio of 15:25:85 PEDOT:PSS:IL:WPU, and the entire mixture was stirred for more than 2 hours. This solution was named “Solution W.”

Solution WG was prepared by adding varying concentrations of glycerol to Solution W, with ratios ranging from 1:1 to 1:300 for test purposes. In a commercial environment, an amount of glycerol may be predetermined by the method set forth herein. Among the tested ratios, Solution WG 1:250 exhibited the most favorable properties for the intended task and was therefore selected. The glycerol-to-solution W ratio of 1:250 was found to provide a good balance between stretchability, conductivity, and ease of fabrication.

A fabrication process was developed for high-density and large-area stretchable electrode arrays in the Solution WGP by optimizing the fabrication steps and parameters. The as-fabricated stretchable sEMG array has a narrow distribution for both the impedance amplitude and the phase angle. Because the entire electrode array (as thin as 70 μm) is made of soft and elastic materials, it can be conformably attached onto human skin allowing for high-density surface electromyography (sEMG) recording even on moving subjects. In addition, the solution processable conductive composite and the array fabrication based on laser patterning allows to obtain custom and large-area sEMG arrays. The typical interelectrode distance for sEMG devices is ~10 mm or larger because the high impedance of rigid electrodes and poor skin contact do not allow for reliable recording at smaller sizes. Because of the flexibility offered by the fabrication method, a device that maps areas up to 19 in² was formed.

Stretchable bioelectronics made with rigid inorganic materials require special structural designs at compromised device densities, whereas Solution WGP can be directly patterned into high-density stretchable arrays, which allows for bioelectronic applications at intricate locations with high precision and customizable patterns.

The stretchable and conductive Solution WG was applied to a glass slide in step 316. After the stretchable and conductive Solution WG was applied to a glass slide and dried in step 320, the electrode array pattern was laser cut in step 320 using a Universal Laser System™ VLS6.75 with a CO2 laser and a High Power Density Focusing Optics (HPDFO) with a focal point of 0.001 inch (25.4 μm) at a power of 1.5 W and a speed of 11% of the instrument's maximum speed. The excess material was then removed, and the electrode array was transferred to the substrate in step 320 (such as Tegaderm™ substrate) via transfer printing. The electrodes were plasma etched for 5 minutes at 120 VAC 60 Hz, 200 m Torr to improve their conductivity and adhesion in step 322. After plasma etching, the electrode array 10 on the substrate 32 was encapsulated in step 324 with a second layer of Tegaderm™ that had been laser patterned with openings 38 (shown in FIG. 1D) to expose the sensing pads 14 for skin contact.

To fabricate the (sEMG) sensor 24 referred to as Solution WGP arrays, step 326 was performed: a gel-like solution of PEDOT:PSS and glycerol in a 1:1 ratio was drop casted onto the exposed electrode areas at a rate of 32 μL/cm2 (31.8 mL/mm2) to improve the contact interface between the sensing electrodes and the skin's topographical features.

To use the sensor 24 on a person, the encapsulated sEMG sensor was thermally annealed in step 328 on a hot plate at 60° C. for 10 minutes before being applied to the skin in step 330. This fabrication process allows for the creation of high-density electrode arrays with a resolution of 100 μm and a device size up to 12 inches, surpassing the limitations of conventional photolithography processes and enabling whole-arm or even whole-body EMG mapping. In one constructed example, the active components made with the composite stretchable conductor, the electrode array 10 having the interconnects 12 and sensing pads 14, were 2.7 μm thick, the substrate layer 32 and the encapsulation layer 34 are each 1.6 μm thick, making the device thickness lower than 6 μm.

An Intan RHD recording system (Intan technologies) was used for EMG recording for electrode validation. The sEMG signal was recorded with a sampling rate of 2500 Hz, a high-pass filter of 10 Hz, a low-pass filter of 500 Hz, and a notch filter of 60 Hz. To obtain sEMG recordings, the reference electrode was placed on the skin, far from the recorded muscle and preferably on a bony prominence, while the two recording electrodes were placed 1 cm apart on the muscle being tested. The electrodes were then connected to a PCB acting as an adapter between the recording chip and the electrode array. Measurement recording was started, and bicep testing was performed as a sample motion. For the bicep testing, the reference electrode was placed on the top of the hand and the two working electrodes were placed 1 cm apart on the bicep brachii. The electrodes were then interfaced with the skin and the Intan, and 5 isometric bicep contractions and 5 bicep flexions were tested. The data was then saved and the RHD file was extracted and moved to the data analysis steps.

Rectangular samples of Solution W and WG were created by drop-casting the solutions onto a substrate, and then left to dry overnight. The dried samples were cut into small rectangular pieces using razor blades, and some were transferred onto AS. Each sample was then mounted into the clips of the UNIvert cell scale stretch station. The stress-strain characteristics of each sample were obtained by stretching them at a 1% strain rate until they fractured. The stress-strain curves revealed that Solution W exhibited a significantly higher strain to failure (~1500%) compared to previously reported PEDOT:PSS-based composites. The addition of glycerol as a disorder-inducing agent resulted in a more uniform and isotropic thin film, as evidenced by SEM cross-sections. Formulations with glycerol-to-Solution W ratios lower than 200 showed no fracturing up to 250% elongation. The same test was repeated with copper wires attached to the clips to measure the resistance. The PalmSens4 was used to collect this data, with the working and reference electrode clips connected to their corresponding stretch station clips while the counter and reference electrodes from the PalmSens were shorted. The stress-strain characteristics and resistance values were collected simultaneously, using the same stretch parameters for each test.

To obtain fatigue properties, the same Solution W and WG samples were mounted onto the stretch station again, using the same 1% strain rate. During the cycling tests, resistance was also measured using the same parameters as in the stress-strain tests. Both freestanding and substrate-supported composites exhibited a decrease in resistance upon cycling. Solution WG250 recovered its initial resistance after about 50 cycles, displaying strain-insensitive resistance.

The PalmSens4 device was used to measure impedance for various electrodes, including Solution W, WP, WG, WGP, and commercially available electrodes. The testing method involved using working, reference, and counter electrodes, with each electrode connected to the PalmSens using alligator clips attached to wires. The working clip was connected to the bottom of the tested electrode, while the reference and counter electrodes were connected through PBS. A platinum electrode was used as the counter electrode, and a silver wire was used as the reference. After connecting the working electrode and submerging the counter and reference electrodes in PBS, impedance measurements were obtained through the creation of a bode plot.

The developed electrodes (Solution WG and WGP) were compared with commercially available electrodes (CE) in terms of sEMG signal quality, adaptability to different skin conditions, and motion artifacts. It was found that for electrode-skin impedance, Solution WG and WGP electrodes showed 70% and 87% lower impedance than CE, respectively. When coated with PEDOT:PSS/Glycerol, the impedance further decreased by 96% and 98% for Solution WP and WGP, respectively.

Determining the signal amplitude and SNR on wrinkled skin, CE showed a 77.2% reduction in signal amplitude and a 38.3% reduction in SNR compared to smooth skin. Solution WG and WGP demonstrated better performance, with only 29.5% and 23% decreases in signal amplitude, and 75% and 53% reductions in SNR, respectively.

On hairy skin, CE exhibited a 60.5% decrease in signal amplitude and a 77.4% decrease in SNR compared to shaved skin. Solution WGP showed minimal changes, with only 6.1% and 0.5% decreases in signal amplitude and SNR, respectively.

During dynamic conditions, CE showed a 44.37% reduction in SNR, while Solution WG and WGP demonstrated only 21.79% and 7.64% reductions, respectively.

These comparative results highlight the superior performance of the developed electrodes, particularly Solution WGP, in terms of signal quality, adaptability to different skin conditions, and resilience to motion artifacts.

The research participants were selected through ResearchMatch and invited to the laboratory where they were asked to flex their biceps while connected to three different types of electrodes: Solution WG, WGP, and commercially available electrodes. Each subject had two electrodes attached to their bicep and a reference electrode attached on the top of their hand, all of which were connected to the INTAN device for reading EMG signals. The experiment consisted of three tests, with each test involving five bicep contractions while using a different electrode. The first test used commercially available electrodes, the second used Solution WG electrodes, and the third used WGP electrodes.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A sensor comprising:
a substrate;
an electrode array disposed on the substrate, the electrode array comprising a conductive polymer, an ionic liquid (IL), a water-based polyurethane matrix (WPU) forming interconnects and sensing pads;
an encapsulation layer disposed on the interconnects of the electrode array;
the interconnects extending between and electrically connecting the sensing pads; and
the pads located on and projecting from a continuous surface of the interconnects opposite the substrate, with the matrix on the pads only being exposed and projecting through openings in the encapsulation layer.

2. The sensor of claim 1 further comprising a coating of the matrix disposed directly on the conductive polymer forming the sensing pads forming a soft and low-impedance interface with skin of less than 70 μm.

3. The sensor of claim 2 wherein the coating comprises poly(3,4-ethylenedioxythiophene):polystyrene sulfonate (PEDOT:PSS) and glycerol.

4. The sensor of claim 1 wherein the substrate comprises acrylic adhesive.

5. The sensor of claim 1 wherein the conductive polymer comprises poly(3,4-ethylenedioxythiophene):polystyrene sulfonate (PEDOT:PSS), and the substrate, the electrode array and the encapsulation layer have a thickness less than 6 μm.

6. The sensor of claim 5 wherein the electrode array further comprises glycerol.

7. The sensor of claim 6 wherein a ratio of glycerol to a mixture of PEDOT:PSS, the ionic liquid and the water-based polyurethane is 1:250.

8. The sensor of claim 7 wherein the encapsulation layer is composed of acrylic adhesive.

9. The sensor of claim 6, wherein:
a ratio of glycerol to the mixture of PEDOT:PSS, the ionic liquid, and the water-based polyurethane is lower than 1:200, exhibiting no fracturing up to 250% elongation; and
the sensing pads are stretchable, thicker than the interconnects, thicker than the encapsulating layer and have a curved periphery.

10. The sensor of claim 1 wherein the conductive polymer, ionic liquid, and water-based polyurethane matrix are in a ratio of 15:25:85, respectively

11. A method of forming an electromyography (EMG) sensor comprising:
forming an electrode solution;
drying the electrode solution to form a dried electrode solution;
forming an electrode array;
transferring the electrode array to a substrate;
plasma etching the electrode array; and
encapsulating the electrode array with an encapsulation layer so sensing pads are exposed at the electrode array to form an encapsulated EMG array.

12. The method of claim 11 wherein:
forming the electrode solution comprises mixing a conductive polymer, an ionic liquid (IL), and a water-based polyurethane matrix (WPU); and
the sensing pads have a low-impedance interface with skin of less than 70 μm.

13. The method of claim 11 wherein forming the electrode solution comprises forming the electrode solution from PEDOT:PSS, ionic liquid and waterborne polyurethane.

14. The method of claim 11 wherein:
forming the electrode solution comprises forming the electrode solution from a mixture of PEDOT:PSS, ionic liquid and waterborne polyurethane and glycerol;
further comprising locating the sensing pads upon an uninterrupted surface of an interconnect, with the sensing pads projecting through openings only in the encapsulation layer;
flexing and stretching the substrate, the encapsulation layer and the sensing pads; and
the substrate, the electrode array and the encapsulation layer have a thickness less than 6 μm.

15. The method of claim 14 wherein a ratio of glycerol to the mixture of PEDOT:PSS, ionic liquid and waterborne polyurethane is 1:250.

16. The method of claim 11 further comprising thermally annealing the encapsulated EMG array and thereafter applying to skin.

17. The method of claim 11 further comprising applying the electrode solution to a slide before drying.

18. The method of claim 11 wherein transferring the electrode array to the substrate comprises transferring the electrode array to the substrate composed of a polyurethane membrane coated with a layer of an acrylic adhesive.

19. The method of claim 11 further comprising applying a PEDOT:PSS and glycerol to coat the sensing pads.

20. The method of claim 19 wherein applying a PEDOT:PSS and glycerol solution comprise applying the PEDOT:PSS and glycerol solution at a ratio of 1:1.

21. A method of forming an electrode comprising:
depositing a mixture of poly(3,4-ethylenedioxythiophene):polystyrene sulfonate (PEDOT:PSS), an ionic liquid (IL), a water-based polyurethane matrix (WPU) and glycerol to form an electrode layer on a first substrate layer that forms interconnects and sensing pads;
encapsulating the interconnects of the electrode layer with an encapsulation layer without encapsulating the sensing pads with the encapsulation layer to form exposed sensing pads;
depositing PEDOT:PSS glycerol on the exposed sensing pads;
locating the sensing pads upon the interconnects, with the sensing pads projecting through openings only in the encapsulation layer;
flexing and stretching the encapsulation layer and the sensing pads;
the electrode layer and the encapsulation layer having a thickness less than 6 μm; and
the sensing pads having a low-impedance interface with skin of less than 70 μm.
